(19) United States
(12) Patent Application Publication
(10) Pub. No.: US 2020/0100654 A1
(43) Pub. Date: Apr. 2, 2020

(54) MINIMAL INVASIVE MEDICAL DEVICE

(71) Applicant: Wipro Limited, Bangalore (IN)

(72) Inventors: Gunalan Selvam, Bangalore (IN); Pavan Misra, Bangalore (IN); Brijesh Babu, Bangalore (IN)

(21) Appl. No.: 16/200,133

(22) Filed: Nov. 26, 2018

(30) Foreign Application Priority Data
Sep. 28, 2018 (IN) 201841036845

(51) Int. Cl. A61B 1/00; A61B 1/06; A61B 1/05

(52) U.S. Cl. CPC A61B 1/00066; A61B 1/00103; A61B 1/00165; A61B 1/0052; A61B 1/05; A61B 1/00105; A61B 1/0676

(57) ABSTRACT

A minimal invasive medical device comprises a reusable portion and a disposable portion connectable by a mechanism. The medical device further includes an actuation unit positioned in the reusable portion. The actuation unit is configured to deflect a tip of the disposable portion. The actuation unit is operable by at least one of a knob and an actuator to deflect the tip of the disposable portion. The configuration of the medical device enables in detaching the disposable portion from the reusable portion, which enables in reusing the medical device by replacing only the disposable portion, which facilitates in ensuring high hygiene and yet minimizing the surgical costs. Also, the medical device provides flexibility in repairing and serviceability.

This application claims the benefit of Indian Patent Application Serial No. 201841036845, filed Sep. 28, 2018, which is hereby incorporated by reference in its entirety.

### FIELD

The present disclosure generally relates to the field of medical devices. Particularly, but not exclusively the present disclosure relates to a minimal invasive medical device. Further embodiments of the present disclosure disclose the minimal invasive medical device, having a reusable portion and a disposable portion.

### BACKGROUND

Diagnosing and treating internal organs of a subject's body may be done in multiple ways or techniques. With advancement in the technology, diagnosis and treatment of internal organs or tissues of subject's body, by a minimal invasive technique has emerged. Presently, the need of the hour is to develop quick and effective techniques for diagnosing and treating problems arising in the subject's physiology or anatomy. Conventionally, invasive techniques were widely and extensively adopted in treating internal organs of the subject's body. Generally, the invasive techniques involve, forming an incision of some size in the vicinity of the organ or tissue to be treated and the required surgery may be carried out therein. Creating this incision in the invasive technique is a tedious and complex process and such incisions would leave a scar on the body of the subject. Additionally, these incisions would require to be sutured which leads to long healing time periods. Such incisions may also potentially lead to acute pain and may lead to probable infections. In order to provide hassle-free treating conditions to the subject, there exists a need to treat the internal organs of the subject's body without or minimum incision.

Considering the above, and with the advent of modern technology, minimal invasive medical devices have been developed. Such devices may be widely used in minimal invasive techniques such as, but not limiting to arthroscopy, laparoscopy, ureteroscopy and the like. Usually, the minimal invasive medical devices may broadly comprise a handle portion and an elongated flexible tubular member. The elongated flexible tubular member may protrude through openings and passageways within the subject's body, to reach the target organ to be treated, without a need of creating the incision. This technique particularly eliminates or minimizes the incisions, which in turn eliminates the use of stitches and hence results in quick recovery of the patient. However, since some components of the minimal invasive device, needs to be inserted into the subject's body, reusability of such devices may become a constraint. In order to reuse such devices, the components which come in contact with the subject's body, need to be sterilized for long hours after each surgical procedure, before using the same for a next use. However, long sterilizing periods may lead to downtime for the practitioners to carry out successive procedures. Furthermore, sterilization delays and costs associated with maintenance of the minimal invasive medical devices have escalated costs for surgical procedures and other medical procedures that utilize similarly configured devices.

Several attempts have been made in the art to mitigate the problems associated with the inappropriate sterilization practices. One such development includes development of disposable minimal invasive medical devices, which are configured to be disposed of after a single use. Since, the minimal invasive medical devices includes complicated electronics, processor units and other complex mechanisms, limiting the device to single use, would escalate the cost of the surgery, which may again demand the utilizers to look back at the non-disposable or sterilizing minimal invasive devices.

The present disclosure is directed to overcome one or more limitations stated above.

### SUMMARY

One or more shortcomings of the conventional minimal invasive medical devices to examine internal organs of the subject are overcome and additional advantages are provided through the present disclosure. Additional features and advantages are realized through the techniques of the present disclosure. Other embodiments and aspects of the disclosure are described in detail herein and are considered a part of the claimed disclosure.

In a non-limiting embodiment of the disclosure, a minimal invasive medical device is disclosed. The minimal invasive device comprises a reusable portion and a disposable portion. The disposable portion is detachably fixed to the reusable portion by a mechanism. Further, the minimal invasive medical device comprises an actuation unit, positioned in the reusable portion. The actuation unit is configured to deflect a tip of the disposable portion. Furthermore, the actuation unit is configured to be operable by at least one of a knob and an actuator.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The accompanying drawings, which are incorporated in and constitute a part of this disclosure, illustrate exemplary embodiments and, together with the description, serve to explain the disclosed principles. The same numbers are used throughout the figures to reference like features and components. Some embodiments of device and/or methods in accordance with embodiments of the present subject matter are now described, by way of example only, and with reference to the accompanying figures, in which:

FIG. 1 illustrates a schematic view of minimal invasive medical device, in accordance with some embodiments of the present disclosure.

FIG. 2 illustrates a schematic front view of a tip of a disposable portion of the minimal invasive medical device of FIG. 1.

FIGS. 3a and 3b illustrates a perspective view of a reusable portion and a latching member of a disposable portion, of the minimal invasive medical device, in accordance with some embodiments of the present disclosure.

FIG. 4 illustrates a perspective view of a mechanism, for detachably connecting the reusable portion and the disposable portion of the minimal invasive medical device, in accordance with some embodiments of the present disclosure.

FIGS. 5a and 5b illustrates a perspective view of a first engageable member and a second engageable member of the mechanism of FIG. 4.

FIGS. 6a-6c illustrates a locking member being operated at different positions during operation of the mechanism, in accordance with some embodiments of the present disclosure.

The figures depict embodiments of the disclosure for purposes of illustration only. One skilled in the art will readily recognize from the following description that alternative embodiments of the structures and methods illustrated herein may be employed without departing from the principles of the disclosure described herein.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiments thereof has been shown by way of example in the drawings and will be described in detail below. It should be understood, however that it is not intended to limit the disclosure to the particular forms disclosed, but on the contrary, the disclosure is to cover all modifications, equivalents, and alternatives falling within the spirit and the scope of the disclosure.

The terms “comprises”, “comprising”, or any other variations thereof, are intended to cover a non-exclusive inclusion, such that device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

Embodiments of the disclosure disclose a minimal invasive medical device. The minimal invasive medical device may comprise a reusable portion and a disposable portion. The disposable portion may be detachably fixed to the reusable portion by a mechanism. The provision of providing the disposable portion and the reusable portion in the minimal invasive medical device allows re-use of the device with minimum or no sterilization. Further, the device facilitates repairing and serviceability, which may effectively reduce the maintenance and other incidental costs and moreover, eliminates the chances of treated organs/tissues from being infected.

The minimal invasive medical device (hereinafter referred as device), comprises a reusable portion and a disposable portion. Further, the device comprises an actuation unit, positioned in the reusable portion. The actuation unit may be configured to deflect a tip of the disposable portion. Further, the actuation unit may be operable by at least one of a knob and an actuator. Additionally, the disposable portion may be detachably fixed to the reusable portion by a mechanism. In an embodiment, detachably fixing the disposable portion to the reusable portion may not be construed as a limitation, as the fixing may be carried out vice versa or by either way.

In some embodiments, the device may comprise a first cable, and a second cable extending from the tip of the disposable portion, a third cable and, a fourth cable extending from the actuation unit in the reusable portion. In an embodiment, free ends of the first cable, the second cable, the third cable, and the fourth cable may be detachably connected by the mechanism. In an embodiment, connecting free ends of the first cable, the second cable, the third cable and, the fourth cable, in turn facilitates in detachably connecting the reusable portion and the disposable portion. The mechanism may comprise a first engageable member configured to the free ends of the first cable and the second cable, and may comprise a second engageable member configured to the free ends of the third cable and the fourth cable. In an embodiment, the first engageable member and the second engageable member facilitates in detachably connecting the first cable, the second cable, the third cable and the fourth cable. Further, the mechanism may also comprise a locking member and a lever, wherein operating the lever may assist in operating the locking member. The locking member is configured to operate between a first position and a second position, thus detachably connecting the first cable, the second cable, the third cable, and the fourth cable, for detachably connecting the disposable portion to the reusable portion.

In the following detailed description, embodiments of the disclosure are explained with reference of accompanying figures that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

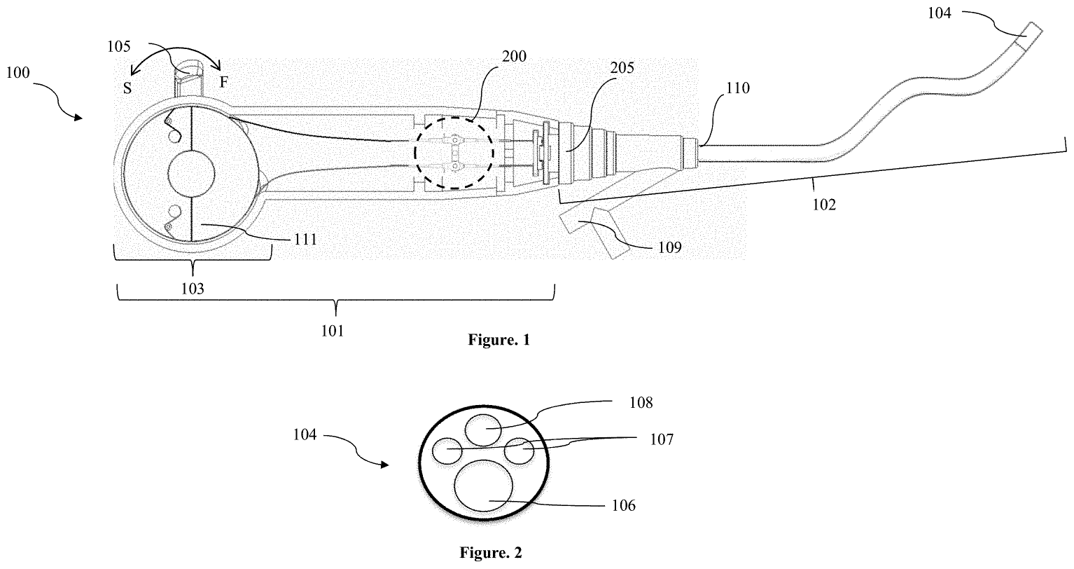

FIG. 1 illustrates a schematic view of a minimal invasive medical device (100), in accordance with some embodiments of the present disclosure.

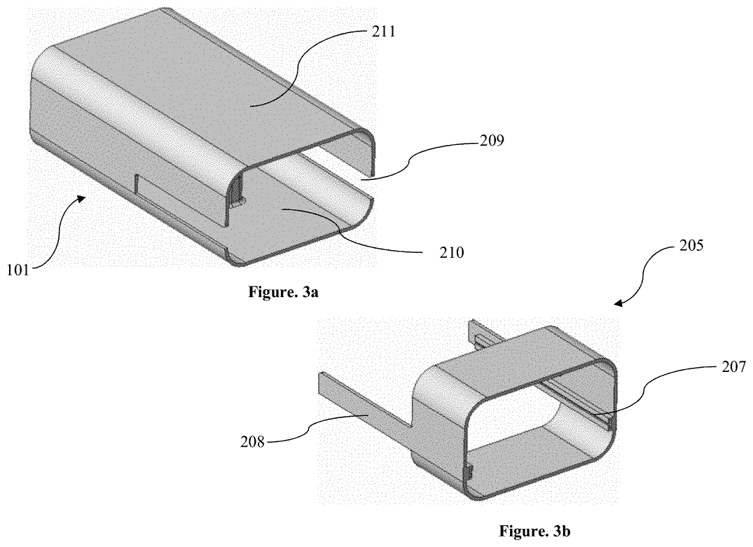

As shown in FIG. 1, the minimal invasive medical device (100) may broadly comprise a reusable portion (101) and a disposable portion (102). In an embodiment, the reusable portion (101) may be a handle assembly capable of being held by a user or an operator, and the disposable portion (102) may be a flexible elongated tubular member. The disposable portion (102) is flexible in order to facilitate the disposable portion (102) to maneuver within passageways in the subject's body to reach a target organ. The disposable portion (102) may be defined with a plurality of channels (106), wherein each of the plurality of channels (106) may be configured to communicate at least one of working fluids, optical fibers, light beam, retrieval baskets, lasers, push-pull cables and the like. Additionally, the disposable portion (102) may comprise an image capturing unit (108) and an illumination unit (107) at a tip (104) [best seen in FIG. 2]. The image capturing unit (108) may be configured to capture images or record the full-motion video images, and the illumination unit (107) may be configured to illuminate for photography and maneuverability within the subject's body.

In some embodiments, the reusable portion (102) may be defined with a cavity (210) for housing a plurality of control modules for regulating an image capturing unit (108), a light source for the illumination unit (107) and feed rate of the optical fiber. In an embodiment, the optical fiber may be a laser fiber. In an embodiment, a plurality of ports (109) may be provided in the reusable portion (101) or the disposable portion (102), wherein the plurality of ports (109) may be configured to provide access to the plurality of channels (106) in the disposable portion (102).

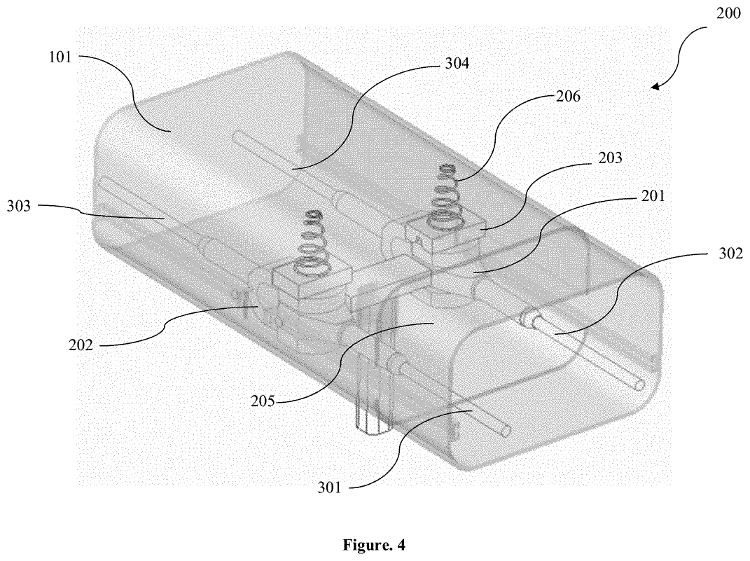

Referring now to FIGS. 3a and 3b, the disposable portion (102) may comprise a latching member (205) (best seen in FIG. 3b), and the latching member (205) may be removably connected to the reusable portion (101). In an embodiment, a proximal end (110) of the disposable portion (102) may be connected to the latching member (205). The latching member (205) may comprise a pair of extensions (208) configured parallel to each other, which may be inserted into a pair of slots (209) (best seen in FIG. 3a) defined on either side of the reusable portion (101). This facilitates in removable connection of the latching member (205) with the reusable portion (101) by at least one of connecting means, such as, but not limiting to a snap-fit connection. Further, the latching member (205) may be configured with a first guideway (207) on an inside portion of each side portion, to facilitate movement of the first cable (301) and the second cable (302), during operation of the device (100).

Further, the device (100) comprises a first cable (301) and a second cable (302), extending from the tip (104) of the disposable portion (102), a third cable (303) and a fourth cable (304) extending from the actuation unit (103), positioned in the reusable portion (101). In an embodiment, free ends of the first cable (301) and the second cable (302) are enclosed within the latching member (205), while the free ends of the third cable (303) and the fourth cable (304) are enclosed within the cavity (210) of the reusable portion (101).The free ends of the first cable (301), the second cable (302), the third cable (303), and the fourth cable (304) may be detachably connectable by the mechanism (200). In an embodiment, detachably connecting the free ends of the cables, facilitates in detachably connecting the reusable portion (101) and the disposable portion (102).

Additionally, the device (100) comprises the actuation unit (103), wherein the actuation unit (103) may be adapted to deflect the tip (104) of the disposable portion (102). In an embodiment, deflecting the tip (104) of the disposable portion (102) may refer to, changing linear configuration of the disposable portion (102) to at least one of a curved profile, an angled profile or a bent profile i.e. the actuation unit (103) may facilitate the tip (104) to be manipulated in various degrees of freedom. However, it may be noted that, adapting the actuation unit (103) to deflect the tip (104) of the disposable portion (102) is an exemplary embodiment, however the same may be adapted to deflect any other portion of the disposable portion (102).

In an embodiment, the actuation unit (103) may be operatively coupled within the reusable portion (101). The actuation unit (103) may comprise at least one of a knob (105) and an actuator [not shown in figures] for operating the actuation unit (103). In an embodiment, the knob (105) may facilitate in operating the actuation unit (103) manually by the user or operator and the actuation of the actuator may facilitate in operating the actuation unit (103) automatically without the need of effort from the user/operator. In an embodiment, the actuator may be an electric motor, such as but not limited to a stepper motor. In some embodiments, the stepper motor may be powered by a power source housed in the device or may be powered by an external power supply means. Further, the actuation unit (103) may comprise a pulley wheel (111), operatively coupled to at least one of the knob (105) and the actuator, wherein operating the at least one knob (105) and the actuator may directly operate the pulley wheel (111). In an embodiment, the outer circumference of the pulley wheel (111) may be configured with a groove for receiving the ends i.e. ends opposite to the free ends of the third cable (303) and the fourth cable (304). The pulley wheel (111) may be configured to pull or push the third cable (303) and the fourth cable (304) based on the direction of operation of at least one of the knob (105) and the actuator. In an embodiment, operating the third cable (303) and the fourth cable (304) may result in operating the first cable (301) and the second cable (302) respectively, since the first cable (301) and the third cable (303) may be detachably connected to form a single unit, and the second cable (302) and the fourth cable (304) may be detachably connected to form a single unit. The first cable (301), the second cable (302), third cable (303) and the fourth cable (304) are illustrated as an exemplary embodiment, and the same may not be construed as a limitation since the cables may be interchangeably connected to each other in the device (100).

By operating the at least one of the knob (105) and the actuator in a first direction (F), the third cable (303) may be under the influence of tension forces and hence the third cable (303) may be pulled towards an end portion of the reusable portion (101). Simultaneously, the fourth cable (304) may be slackened due to tensioning of the third cable (303) and the fourth cable (304) may be pushed away from the end portion of the reusable portion (101). In an embodiment, tensioning and slacking the third cable (303) and the fourth cable (304) respectively, may result in tightening and slacking of the first cable (301) and the second cable (302) respectively, which may result in deflecting the tip (104) of the disposable portion (102) in a first direction. Further, operating the at least one of the knob (105) and the actuator in the second direction (S), the fourth cable (304) may be under the influence of tension forces and hence the fourth cable (304) may be pulled towards an end portion of the reusable portion (101). Simultaneously, the third cable (303) may be slackened due to the tensioning of the fourth cable (304) and the third cable (303) may be pushed away from the end portion of the reusable portion (101). In an embodiment, tensioning and slacking the fourth cable (304) and the third cable (303), may result in tightening and slacking of the second cable (302) and the first cable (301) respectively, which may result in deflecting the tip (104) of the disposable portion (102) in a second direction, opposite to the first direction.

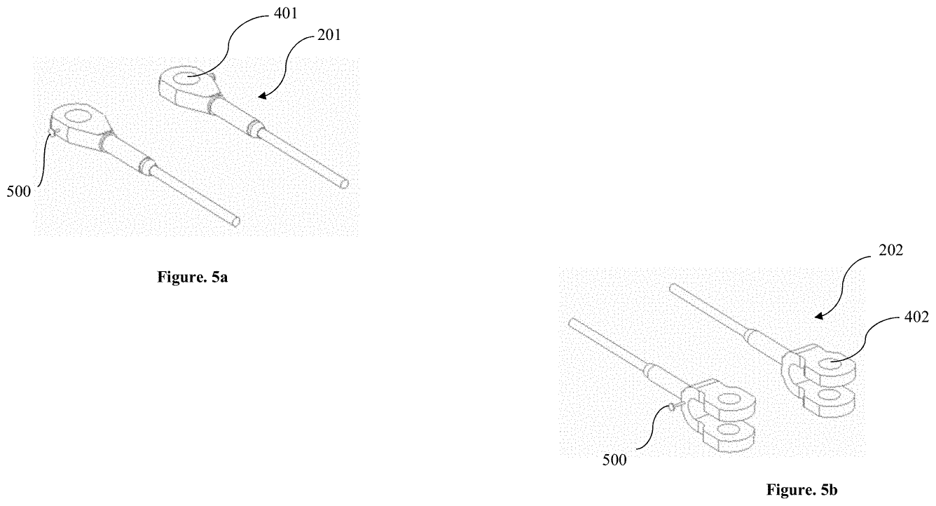

FIG. 4 illustrates perspective view of a mechanism (200), for detachably connecting the reusable portion (101) and the disposable portion (102), in accordance with some embodiments of the present disclosure.

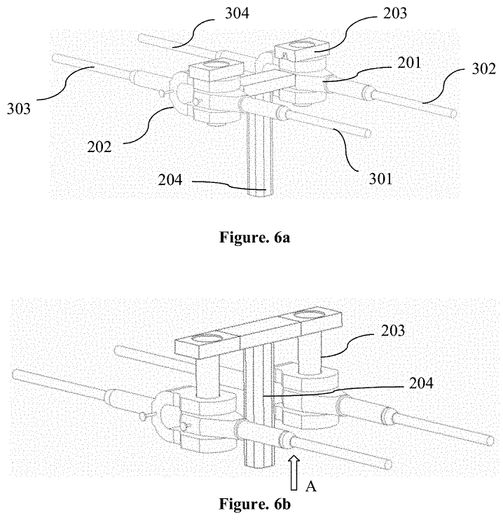

As illustrated in FIG. 4, the mechanism (200) comprises a first engageable member (201) configured at the free end of the first cable (301) and the second cable (302), and a second engageable member (202) configured at the free end of the third cable (303) and the fourth cable (304). As illustrated in FIG. 5a, the first engageable member (201) may be configured with a loop portion, in which a first locking cavity (401) may be defined. Further, as seen in FIG. 5b, the second engageable member (202) may be configured with a U-shaped portion, having a pair of prongs, in which a second locking cavity (402) may be defined. The first engageable member (201) and the second engageable member (202) may be configured with a pin (500) protruding outwardly on either side. In an embodiment, the pin (500) facilitates in guiding and sliding of the first engageable member (201) and the second engageable member (202) during operation of the actuation unit (103). In an embodiment, the second engageable member (202) may be adapted to receive the first engageable member (201), to align the first locking cavity (401) with the second locking cavity (402).

Further, the mechanism (200) may comprise a locking member (203), which is receivable by the first locking cavity (401) and the second locking cavity (402) of the first engageable member (201) and the second engageable member (202) respectively. In an embodiment, the locking member (203) may be operable between a first position and the second position. The locking member (203) may comprise a top portion and a body portion. In an embodiment, the top portion may be defined with a flat profile, and the body portion may be defined of any geometrical profile. Further, a resilient member (206) may be provided on the top portion of the locking member (203), wherein the resilient member (206) may be configured to store and release the energy during displacement of the locking member (203) from the first position and the second position. In an embodiment, the locking member (203) may be configured to displace within the first locking cavity (401) and the second locking cavity (402) of the first engageable member (201) and the second engageable member (202) respectively. Furthermore, the mechanism (200) comprises a lever (204), operatively coupled to the locking member (203). The lever (204) may comprise a flat head portion and a body portion. The lever (204) may be operatively coupled to the locking member (203) and the lever (204) facilitates in operating the locking member (203) between the first position and the second position, during operation of the mechanism (200). In an embodiment, the body portion of the lever (204) may be configured with, but not limited to a cylindrical configuration.

In some embodiments, the mechanism (200) may be positioned in the reusable portion (101). The locking member (203) is adhered within the cavity (210) of the reusable portion (101) via a resilient member (206). In an embodiment, the resilient member (206) may be a deformable member, such as, but not limited to a coil spring, a helical spring and the like. The resilient member (206) may be positioned within second guideways (not shown in figures), provided on the inside portion of a top surface (211) of the reusable portion (101). Further, the resilient member (206) may be configured to slide within the second guideways, during operation of the invasive medical device (100). Further, the lever (204) may be configured in manner such that, one part of a body portion of the lever (204) protrudes outside the reusable portion (101) and rest of the body portion along with the head portion is within the cavity (210) of the reusable portion (101). In an embodiment, one part of the body portion of the lever (204) facilitates the user/operator to displace the locking member (203) between the first position and the second position, facilitating in engaging or detaching the disposable portion (102) and the reusable portion (101).

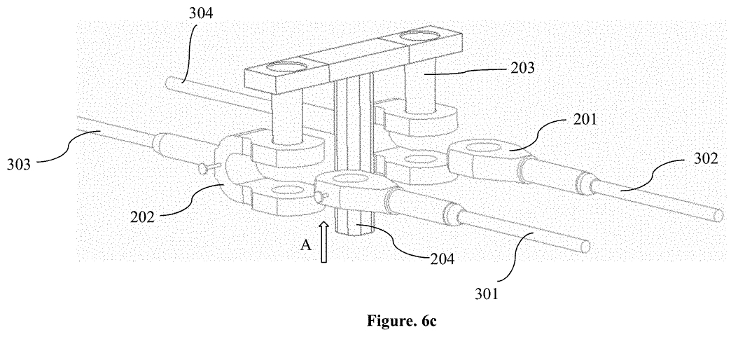

FIG. 6a-6c illustrates a locking member (203) being operated at different positions during operation of the mechanism (200), in order to detach and engage the disposable portion (102) and the reusable portion (101), in accordance with some embodiments of the present disclosure.

As illustrated in FIG. 6a, the locking member (203) is at the first position and resides within the first locking cavity (401) and the second locking cavity (402) of the first engageable member (201) and the second engageable member (202) respectively, at the first position. The resilient member (206) at the first position is in a free state wherein, the locking member (203) is held in place by the force of the resilient member (206). This allows the locking member (203), in engaging the free ends of the first cable (301), the second cable (302), the third cable (303), and the fourth cable (304) respectively, which in turn facilitates in engaging the disposable portion (102) to the reusable portion (101) of the device (100).

As illustrated in FIG. 6b, the locking member (203) is at the second position. In an embodiment, force may be applied by the user or operator to one part of the body portion of the lever (204) in a longitudinal direction (A). Due to the force applied, the head portion of the lever (204) may be operatively coupled with the top portion of the locking member (203), thus displacing the locking member (203) from the first position (best seen in FIG. 6a) to the second position (as seen in FIG. 6b). While, displacing the locking member (203) from the first position to the second position, the resilient member (206) may contract, thus storing the energy in form of potential energy. This potential energy may be exerted back on the locking member (203) to displace the locking member (203) back from the second position to the first position, upon release of the force applied on the body portion of the lever (204). In an embodiment, the locking member (203) in the second position resides away from the first locking cavity (401) and the second locking cavity (402). This facilitates in detaching the free ends of the first cable (301), and the second cable (302) from the third cable (303) and the fourth cable (304) respectively (best seen in FIG. 6c). In an embodiment, detaching the free ends of the cables, may facilitate in detaching the latching member (205) from the reusable portion (101), which in turn results in detaching the disposable portion (102) from the reusable portion (101).

In an embodiment, the mechanism (200) may be operated for detaching the disposable portion (102) from the reusable portion (101), by applying necessary force in a longitudinal direction (A) to the body portion of the lever (204), to operatively couple the top portion of the locking member (203), in order to displace the locking member (203) from the first position to the second position. At the second position, the locking member (203) may reside away from the first locking cavity (401) and the second locking cavity (402). This disengages the free ends of the first cable (301), the second cable (302) from the third cable (303) and the fourth cable (304) respectively, which may facilitate in detaching the latching member (205) of the disposable portion (102) from the reusable portion (101), resulting in detaching the disposable portion (102) from the reusable portion (101).

In an embodiment, the mechanism (200) may be operated for engaging the disposable portion (102) to the reusable portion (101). Initially, the locking member (203) may be displaced to the second position. The latching member (205) of the disposable portion (102) may be connected to the reusable portion (101), which results in aligning the first engageable member (201) of the first cable (301) and the second cable (302) with the second engageable member (202) of third cable (303) and the fourth cable (304) respectively. Further, releasing the force applied on the lever (204) member results in displacing the locking member (203) from the second position to the first position due to the force exerted by the resilient member (206). The locking member (203) in the first position, resides within the first engageable member (201) and the second engageable member (202). This facilitates in engaging the first cable (301) and the second cable (302) with the third cable (303) and the fourth cable (304) respectively, which in turn facilitates in engaging the disposable portion (102) with the reusable portion (101).

In an embodiment, the minimal invasive medical device may be such as, but not limited to an ureteroscope, an endoscope, a hysteroscope, a bronchoscope, a cystoscope and the like.

Advantages of the embodiment of the present disclosure are illustrated herein.

In an embodiment, the present disclosure discloses a minimal invasive medical device with a disposable portion and a reusable portion, which facilitates in using the same device for successive surgical procedures with minimum cost i.e. by replacing disposable portion only.

In an embodiment, the minimal invasive medical device facilitates in repairing and serviceability of the device.

In an embodiment, actuation unit of the minimal invasive medical device may be automated by an actuator such as an electric motor for precise deflection of the tip of the disposable portion.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A minimal invasive medical device, comprising:
a reusable portion;
a disposable portion, detachably fixed to the reusable portion by a mechanism;
an actuation unit positioned in the reusable portion, the actuation unit is configured to deflect a tip of the disposable portion, wherein the actuation unit is operable by at least one of a knob and an actuator.

2. The minimal invasive medical device as claimed in claim 1, wherein the disposable portion is a flexible elongated tubular member.

3. The minimal invasive medical device as claimed in claim 1, wherein the disposable portion is defined with a plurality of channels, each for communicating at least one of working fluid, an optical fiber, or a light beam, and further comprising an image capturing unit and an illumination unit positioned in one of the plurality of channels.

4. The minimal invasive medical device as claimed in claim 1, wherein the reusable portion is a handle defining a cavity.

5. The minimal invasive medical device as claimed in claim 4, wherein the cavity houses a plurality of control modules for regulating an image capturing unit, an illumination unit, and an optical fiber.

6. The minimal invasive medical device as claimed in claim 1 further comprising a first cable and a second cable extending from the tip of the disposable portion, a third cable and a fourth cable extending from the actuation unit, wherein free ends of the first cable, the second cable, the third cable and the fourth cable are detachably connectable by the mechanism.

7. The minimal invasive medical device as claimed in claim 1, wherein the actuation unit comprises a pulley wheel coupled to the at least one of the knob and the actuator.

8. The minimal invasive medical device as claimed in claim 6, wherein the third cable and the fourth cable are attached to a pulley wheel, and the pulley wheel pulls or pushes the third cable and the fourth cable based on direction of operation of the at least one of the knob and the actuator.

9. The minimal invasive medical device as claimed in claim 1 comprises a latching member removably connected to the reusable portion, wherein the latching member houses a first cable and a second cable.

10. The minimal invasive medical device as claimed in claim 9, wherein the latching member comprises a first guideway at each side portion, to facilitate movement of a first cable and a second cable

11. The minimal invasive medical device as claimed in claim 1, wherein the mechanism comprises:
a first engageable member configured at the free end of a first cable and a second cable wherein the first engageable member is defined with a first locking cavity;
a second engageable member configured at the free end of a third cable and a fourth cable, wherein the second engageable member is defined with a second locking cavity, and the second engageable member is adapted to receive the first engageable member, to align the first locking cavity with the second locking cavity;
a locking member receivable by the first locking cavity and the second locking cavity, wherein the locking member is operable between a first position and a second position; and
a lever, operatively coupled to the locking member, wherein actuation of the lever operates the locking member between the first position and the second position.

12. The minimal invasive medical device as claimed in claim 11, wherein the mechanism comprises a resilient member at a top portion of the locking member.

13. The minimal invasive medical device as claimed in claim 12, wherein the resilient member is configured to store energy during displacement of the locking member from the first position to the second position, and release the energy to displace the locking member from the second position to the first position.

14. The minimal invasive medical device as claimed in claim 12, wherein the resilient member is adapted to slide along a second guideway provided on an inner top surface of the reusable portion.

15. The minimal invasive medical device as claimed in claim 11, wherein the locking member in the first position resides within the first locking cavity and the second locking cavity.

16. The minimal invasive medical device as claimed in claim 11, wherein the locking member in the second position resides away from the first locking cavity and the second locking cavity.
